(19) United States
(12) Patent Application Publication
(10) Pub. No.: US 2005/0037945 A1
(43) Pub. Date: Feb. 17, 2005

(54) Fragrance composition to be distributed by an aerosol generator

(76) Inventors: Hans Rudolf Gygax, Bad-Ragaz (CH); Thierry Audibert, Saint Leu la Foret (FR)

Correspondence Address: NORRIS, MCLAUGHLIN & MARCUS, 875 THIRD AVE, 18TH FLOOR, NEW YORK, NY, 10022, US

(21) Appl. No.: 10/476,479

(22) PCT Filed: Apr. 23, 2002
(86) PCT No.: PCT/CH02/00223

(30) Foreign Application Priority Data
May 30, 2001 (EP) 01810432.3

(51) Int. Cl. A61K 7/46

(52) U.S. Cl. 512/1

(57) ABSTRACT

The present invention relates to fragrance compositions to be distributed by an aerosol generator. Said fragrance composition comprises 30% by weight or more of a component of the odour class A, wherein said components are characterised by a sensory threshold concentration that is 1 ng/l or higher and its vapour pressure that is 10 μg/l or higher.

The present invention relates to fragrance compositions for distribution by an aerosol generator.

During the past years the products for air-freshening and fragrancing experienced an important growth in sales. Various device technologies were used to provide a scented environment ranging from simple air-circulation systems to devices using heat and/or carrier materials (e.g. gel and candles). A problem of said device technologies (“classical technologies”) was that the odour quality obtained was not sufficient.

In providing fragrance compositions with desirable accords for these “classical technologies”, the perfumer should ensure that the correct balance of volatile “Top Notes” and less volatile “Bottom Notes” are presented in the vapour phase over the lifetime of a product. Accordingly, the perfumer must consider the vastly different evaporation rates of fragrance ingredients used to make up fragrance compositions. One method employed by perfumers to ensure that sufficient Bottom Notes are expressed in the vapour phase is to overdose on the Bottom Notes and also to use solvents to help lift the Bottom Notes into the vapour phase in order to blend with the Top Notes and achieve a balanced fragrance accord. The terms “Top Note” and “Bottom Note” are generally recognised in the perfumery art and the term “Bottom Note” would include fragrance compounds having a vapour pressure below 10 micrograms/litre.

Whereas this approach enables the perfumer to produce pleasant fragrance accords, it is also rather expensive because Bottom Notes tend to be expensive. Furthermore, the use of large amounts of solvent is undesirable if the fragrance composition is intended to be delivered from miniaturised or portable devices, where volume efficiency is an important factor.

Aerosol generators offer an advantage over the “classical technologies” in that they are adapted to positively express both Top Notes and the less volatile Bottom Notes into the vapour phase. Essentially aerosol generators take the consideration of different evaporation rates of fragrance ingredients out of the equation when designing fragrance accords. However, the use of aerosol generators may create additional problems for the perfumer. Firstly, fragrance compositions created for “classical technologies” may not be suitable for dispensing from aerosols because the optimal balance of Top and Bottom Notes taking into account the evaporation behaviour of the components, may be distorted and result in an unacceptable fragrance accord heavy in Bottom Notes. Secondly, the use of high viscosity fragrance ingredients cause problems in aerosol generators as has been reported in the art(see for example WO 00/47335). In order to overcome the problems with viscosity, it is necessary to include large volumes of solvents such as triethylene glycol and ethanol, and this has the disadvantage, at least, of volume efficiency as mentioned above. Still further, in order to ensure that the correct amount of solvents are employed to attain the desired viscosity, manufacturers of fragrance compositions for use in aerosol generators have to undertake an iterative process requiring a number of assays to obtain the desired viscosity. This is time consuming and adds complexity to the manufacturing process that ultimately may affect the cost of such fragrance compositions.

Accordingly, there remains a need to provide fragrance compositions suitable for dispensing from aerosol generators and for reliable and cost effective methods of producing same.

Surprisingly applicant has now found that a fragrance composition comprising 30% by weight or more of odour class A components may be dispensed by an aerosol generator, wherein said odour class A components are characterised by a Sensory Threshold Concentration that is 1 ng/l or higher and a vapour pressure that is 10 μg/l or higher.

The invention therefore provides in a first aspect a fragrance composition comprising 30% by weight or more of odour class A components wherein said components are characterised by a Sensory Threshold Concentration that is 1 ng/l or higher and a vapour pressure that is 10 μg/l or higher.

In a further aspect the present invention refers to fragrance compositions comprising in addition up to 70% by weight of odour class B components. Components of an odour class B have a Sensory Threshold Concentration smaller than 1 ng/l, and a vapour pressure smaller than 10 μg/l.

The Sensory Threshold Concentration is defined as the concentration of an odorous material for which the probability of detection of the stimulus is 0.5 (that is 50% above chance, by a given individual, under the condition of the test) The Sensory Threshold Concentration can be measured by standard methods, for example described in ASTM E1432-91 and is measured either by olfactometry means or by using sniff-bottles allowing panellists to smell the presented headspace. It is also possible to smell the presented odour in a sequential process.

Certain natural fragrant raw materials, such as essential oils, resinoids, and absolutes generally comprise several components. Whether said fragrant raw material belongs to the odour class A or odour class B is determined by analysis of the five components present in the highest concentration. From these five components the sensory threshold concentration and the vapour pressure has to be determined. If all five components taken alone are in one odour class then the fragrant raw material is classified in the same class. To give an example, in lavender oil (i.e. Lavendin essence grosso) the five components present in the highest concentration are linalyl acetate (about 34.4% wt), linalool (about 29.3% wt), campher (about 7.5% wt), eucalyptol (about 5.2% wt) and terpinen-4-ol (about 2.3% wt). All five components fall within the definition of the odour class A. Thus, lavender oil an essential oil belongs according to the definition of the present invention to components of the odour class A.

By categorising fragrant components as class A or class B and ensuring 30% by weight or more of class A components in a composition, fragrance compositions according to the present invention meet all requirements concerning viscosity, necessary for the filling and dispensing from aerosol generators. Furthermore, the time-consuming iterative process of carrying out a large number of assays to obtain the desired viscosity of a fragrance composition by adding a solvent or carrier fluid without changing its odour impact is rendered unnecessary. This constitutes a great advantage of the present invention.

Thus, the fragrance composition according to the present invention may be formulated on the basis of its desired odour and not on any viscosity considerations, which is an advantage for perfumers and manufacturers of products containing said fragrance compositions.

The presence of 30% wt or more of components of the odour class A in a fragrance composition according to the present invention ensures good sprayability of the fragrance composition even if other components, such as components of the odour class B are present.

In a preferred embodiment the fragrance composition of the present invention comprises at least 50%, preferably 75% by weight of the components of the odour class A and up to 50% by weight, most preferably up to 25% of the components of the odour class B.

Components of the odour class A are preferably selected from the group of allylamylglycolate, allyl caproate, alpha pinene, beta pinene, terpineol, alpha terpineol, amyl acetate, benzyl acetate, benzyl methyl ether, borneol, butyl butyrate, cis-3-hexenol, cis-3-hexenylacetate, citronellal, citronellol, citronellyl acetate, ethyl isobutyrate, cyclal C, lemarome, ethyl butyrate, fructone, ethyl caprylate, hexyl acetate, linalool, linalyl acetate, diethyl maleate, limonene, phenyl propyl alcohol, tetrahydrolinalol, gardenol, styrallyl acetate, isomenthone, menthol, geraniol, geranyl acetate, melonal, phenyl ethyl alcohol, rose oxyde, nonyl aldehyde campher, eucalyptol and terpnen-4-ol.

Components of the odour class B are preferably selected from the group of hedione, methyl octine carbonate, damascenone, damascone delta, damascone alpha, undecatriene, ambrettolide, ambroxan, ambrofix, ambrettone, vanilline, ethyl vanilline, eugenol, evernyl, cashmeran, ethylene brassylate, galaxolide, fixolide, tonalide, crysolide, celestolide, bacdanol and cedryl acetate.

In a preferred embodiment of the present invention at least one component of the odour class A or one component of the odour class B is a fragrance raw material of natural origin, i.e. which is not obtained synthetically.

Fragrance raw materials of natural origin are well known for the person skilled in the art and may be selected from the list of ambrette seed absolute, anise oil, artemisia oil, basil oil, bay oil, bensoin siam resinoid, bergamot oil, birch tar oil, calamus oil, cassia oil, cedarwood oil, cinnamon bark oil, citronella oil, civet absolute, clove oil, cumin oil, cypress oil, dill weed oil, elemi resinoid, estragon oil, eucalyptus oil, fennel oil, galbanum resinoid, ginger oil, grapefruit oil, hyacinth absolute, jasmine absolute, lavender oil, lemon oil, cubeba oil, nutmeg oil, marjoram oil, mandarin oil, mimosa concrete, neroli oil, oakmoss resinoid, olibanum resinoid, orange oil, origanum oil, orris oil, oamnathus absolute, patchouli oil, pepper oil, peppermint oil, pimento oil, pine needle oil, rosemary oil, rose oil, rosewood oil, sandalwood oil, thyme oil, tuberose absolute, vetiver oil, and ylang ylang oil.

When distributed by aerosol generators the fragrance compositions according to the present invention have a constant and stable olfactive quality over the life time of the product. Even the so-called Top Notes are still present over the whole life time. Top Notes as used herein means high volatile compounds, e.g. compounds having a vapour pressure above about 1000 μg/l.

Optionally the fragrance composition may contain fillers such as propylengylcol methyl ether, dipropylenglycol, triethylenglycol, ethanol, dipropylenglycol, diethylphtalaten and carbitol. These materials are not included in the fragrance composition for their impact on the odour of the fragrance composition and are therefore not considered as materials of either an odour class A or B for the purpose of this invention.

Other excipients such as insect repellents and antibacterials, for example Bronopol® may be present in the fragrance composition.

The fragrance composition according to the present invention may be used in all kinds of aerosol generators.

A variety of aerosol generators have been used to distribute fragrance compositions. One method to vaporise a fragrance composition is by a piezoelectric spraying system. Said piezoelectric spraying system generates droplets through actuated piezo-membranes. Examples for such piezoelectric spraying system are disclosed in WO 00/47335 and U.S. Pat. No. 6,014,970. Another aerosol generator is an electrospray device, which uses an electric field to generate small droplets. Such generators are described for example in EP 194074 and WO94/12285. Another aerosol generator, a so-called “Venturi device” which is also known under the name “Indigo” splits a liquid mechanically into small droplets. Such a device is capable of generating very small droplets (<10 μm) which allow a fast transition of the aerosolised fragrance portion into the gas phase. A “Venturi device” is described, for example, in WO 99/49904.

Especially preferred are aerosol generators which are spraying systems such as piezoelectric spraying systems as described in WO00/47335 and U.S. Pat. No. 6,014,970, which are hereby incorporated herein by reference, electrospray devices as described in EP 194074 and WO94/12285, which are hereby incorporated herein by reference, or venturi devices as described in WO99/49904, which is incorporated herein by reference. Preferably, the fragrance composition according to the present invention is distributed at ambient temperature.

Fragrance compositions may be filled into devices containing aerosol generators. For example a fragrance composition in liquid form may be poured into a suitable receptacle provided in an aerosol generator device. Alternatively, fragrance compositions may be filled into a cartridge, which in turn may be adapted to be received in an aerosol-generator device, optionally in a refillable manner. Such cartridges may take any convenient form. For example, the cartridge may be in the form of an air-less bag, e.g. similar to the airless bags employed in ink-jet printer cartridges.

Fragrance compositions according to the present invention may be used in a wide variety of air-freshening and fragrancing products. For example, products used for scenting large spaces such as living spaces, hotel rooms or lobbies, or the like, e.g. household or building ventilation and air-conditioning systems; multi-media products, e.g. home entertainment devices, scented cinema devices, computer and internet game devices; and devices used to scent cars and other means of transportation.

Because the fragrance compositions can be employed without using large volumes of solvents or fillers, they are particularly suitable for filling devices which are designed to be miniaturised or portable, or which are intended to incorporate a plurality of small-volumed, multi-scent cartridges where volume efficiency is important.

There now follows a series of examples that illustrate the invention.

### EXAMPLE 1

| | weight % |
|---|---|
| INDOLE PUR²⁾ | 0.14 |
| VANILLINE²⁾ | 0.29 |
| ACET GERANYLE SYNT¹⁾ | 1.43 |
| RADJANOL SUPER²⁾ | 1.43 |
| THIBETOLIDE²⁾ | 1.43 |
| CYCLOHEXAL²⁾ | 2.86 |
| GARDENOL¹⁾ | 2.86 |
| HELIOTROPINE CRIST²⁾ | 2.86 |
| SALICYLATE BENZYLE²⁾ | 2.86 |
| EUGENOL PUR²⁾ | 3.57 |
| TETRAHYDRO LINALOL¹⁾ | 3.57 |
| CITRONELLOL EXTRA¹⁾ | 4.29 |
| LILIAL²⁾ | 4.29 |
| YLANG YLANG ESS 3 ORPUR¹⁾ | 5.71 |
| ISO E SUPER²⁾ | 5.71 |
| ALC PHENYL ETHYLIQUE¹⁾ | 7.14 |
| HEDIONE²⁾ | 7.14 |
| TERPINEOL PUR¹⁾ | 8.57 |
| IONONE BETA²⁾ | 10.71 |
| PROPYLENE GLYCOL METHYL ETH | 23.14 |
| Total | 100.00 |

### EXAMPLE 2

| | |
|---|---|
| N112²⁾ | 0.05 |
| OXANE 50%/CITR²⁾ | 0.10 |
| OCTINE CARBONATE METHYLE²⁾ | 0.10 |
| ACET CITRONELLYLE¹⁾ | 0.20 |
| DAMASCENONE²⁾ | 0.20 |
| HEXENOL-3-CIS¹⁾ | 0.20 |
| ALD C 9 NONYLIQUE FCC¹⁾ | 0.40 |
| MENTHOL NATUREL¹⁾ | 0.40 |
| AMBRETTOLIDE²⁾ | 0.50 |
| ISOMENTHONE DL¹⁾ | 0.50 |
| METHYL-2-BUTYRATE ETHYLE¹⁾ | 0.50 |
| ROSE OXYDE CO¹⁾ | 0.50 |
| UNDECATRIENE²⁾ | 0.50 |
| BUTYRATE AMYLE¹⁾ | 1.00 |
| PECHE PURE²⁾ | 1.00 |
| CYCLAL C¹⁾ | 1.50 |
| LABIENOXIME 1%/CQS²⁾ | 1.50 |
| LEMAROME N¹⁾ | 1.50 |
| BUTYRATE ETHYLE¹⁾ | 2.00 |
| FRUCTONE¹⁾ | 2.00 |
| BUTYRATE DIMETHYL BENZ CARB¹⁾ | 2.50 |
| CAPRONATE ALLYLE¹⁾ | 3.00 |
| CARPRONATE ETHYLE¹⁾ | 3.00 |
| ACET HEXYLE¹⁾ | 5.00 |
| CITRONELLOL EXTRA¹⁾ | 5.00 |
| HEDIONE²⁾ | 5.00 |
| ROSOFLOR 2¹⁾ | 7.50 |
| LINALOL SYNT¹⁾ | 15.00 |
| ORANGE TERPENES DIST¹⁾ | 19.35 |
| MALONATE DIETHYLE¹⁾ | 20.00 |
| Total | 100.00 |

¹⁾component of the odour class A
²⁾component of the odour class B
All components above are commercially available at Givaudan SA, Vernier, Switzerland.

1. A fragrance composition comprising 30% by weight or more of odour class A components, wherein said components are characterised by a Sensory Threshold Concentration that is 1 ng/l or higher and a vapour pressure that is 10 μg/l or higher.

2. A fragrance composition according to claim 1 comprising at least 50% by weight of odour class A components.

3. A fragrance composition according to any of the preceding claims comprising at least 75% by weight of odour class A components.

4. A fragrance composition according to any of the preceding claims additionally comprising up to 70% by weight of an odour class B component, wherein said components are characterised by a Sensory Threshold Concentration that is smaller than 1 ng/l and a vapour pressure that is smaller than 10 μg/l.

5. A fragrance composition according to claim 4 comprising up to 50% by weight of odour class B components.

6. A fragrance composition according to claim 4 comprising up to 25% by weight of odour class B components.

7. A fragrance composition according to any of the preceding claims, wherein at least one component of the odour class A is a natural fragrance raw material.

8. A fragrance composition according to any of the claims 4 to 7, wherein at least one component of the odour class B is a natural fragrance raw material

9. A fragrance composition according to claim 1 distributed by an aerosol generator.

10. Use of a fragrance composition according to any of the preceding claims in a device using an aerosol generator for dispensing of said fragrance composition.

11. Use of a fragrance composition according to claim 10, wherein the aerosol generator is selected from a piezoelectric spraying system, an electrospray device, and a venturi device.

12. Use of a fragrance composition according to claim 10 or claim 11 at ambient temperature.

13. A device containing an aerosol generator for dispensing a fragrance composition containing a composition as defined in any of the claims 1 to 9.

14. A device according to claim 13 containing an aerosol generator selected from the group consisting of a piezoelectric spraying system, an electrospray device, and a venturi device.

15. An air-freshening or air-fragrancing device according to claim 14 selected from multi-media devices, home entertainment devices, scented cinema devices, computer and internet game devices and devices used to scent cars and other means of transportation.

16 A cartridge containing a fragrance compositions as defined in any of the claims 1 to 9.

17 A cartridge according to claim 16 comprising an air-less bag.

18 A cartridge for use in a device as defined in claim 14 or 15.

19 A method of manufacturing a device comprising an aerosol generator comprising the step of filling the device with a fragrance composition as defined in any of the claims 1 to 9.

20 A method of manufacturing a fragrance composition comprising the step of selecting 30% by weight or more of odour class A components, wherein said components are characterised by a Sensory Threshold Concentration that is 1 ng/l or higher and a vapour pressure that is 10 μg/l or higher, and optionally blending these components with one or more fragrance components or excipients used in fragrance compositions.
